(19) United States
(12) Patent Application Publication
(10) Pub. No.: US 2007/0129618 A1
(43) Pub. Date: Jun. 7, 2007

(54) Blood parameter testing system

(76) Inventors: Daniel Goldberger, Boulder, CO (US); Eric Shreve, Louisville, CO (US); Wayne Siebrecht, Golden, CO (US); Benny Pesach, Rosh Haayin (IL); Gidi Pesach, Kfar Vitkin (IL); Gabby Bitton, Jerusalem (IL); Ron Nagar, Tel Aviv (IL)

Correspondence Address: PATENTMETRIX, 14252 CULVER DR. BOX 914, IRVINE, CA, 92604, US

(21) Appl. No.: 11/157,110

(22) Filed: Jun. 20, 2005

(51) Int. Cl. A61B 5/05; A61B 5/00

(52) U.S. Cl. 600/345; 600/347; 600/365

(57) ABSTRACT

A substantially automatic blood parameter testing apparatus for obtaining a blood sample and determining the concentration of at least one analyte is connected to a venous or arterial access line and further comprises a pump fixedly attached to a tube originating from the vascular access point; a valve fixedly attached to the tube and located above the pump mechanism; at least one measurement element; a needleless port; and an electronic meter. In addition, the automated blood parameter testing apparatus may be integrated into a complete system, further including a monitor and a central monitoring station.

### FIELD OF THE INVENTION

The present invention relates generally to systems and methods for automatically measuring physiological parameters and blood constituents, and in particular, to a method and system for automated blood glucose measurement. In addition, the present invention relates to improved methods of using sensors in operating the automated blood parameter testing system.

### BACKGROUND OF THE INVENTION

Patient blood chemistry and monitoring of patient blood chemistry are important diagnostic tools in patient care. For example, the measurement of blood analytes and parameters often give much needed patient information in the proper amounts and time periods over which to administer a drug. Blood analytes and parameters tend to change frequently, however, especially in the case of a patient under continual treatment, thus making the measurement process tedious, frequent, and difficult to manage.

For example, diabetes mellitus can contribute to serious health problems because of the physical complications that can arise from abnormal blood glucose levels. In the United States alone, it is estimated that over 11 million people suffer from diabetes. The two most common forms of diabetes are Type I, juvenile-onset, and Type II, adult-onset. Type I diabetes destroys the vast majority of the insulin-producing beta cells in the pancreas, thus forcing its sufferers to take multiple daily insulin injections. Type II diabetes is usually less severe than Type I, causing a decreased level of endogenous insulin production in the body, and can often be controlled by diet alone.

The body requires insulin for many metabolic processes; it is chiefly important for the metabolism of glucose. If normal blood glucose levels are maintained throughout the day, it is believed that many of the physical complications associated with diabetes could be avoided. Maintaining a consistent and normal blood glucose level is an arduous task as the diabetic's blood glucose level is prone to wide fluctuations, especially around mealtime. Many diabetics are insulin dependent and require routine and frequent injections to maintain proper blood glucose levels.

Unlike the normal functioning of the body's glucose control systems, injections of insulin do not incorporate feedback mechanisms. Controlling glucose levels therefore requires continuous or frequent measurements of blood glucose concentration in order to determine the proper amount and frequency of insulin injections. The ability to accurately measure analytes in the blood, particularly glucose, is important in the management of diseases such as diabetes as described above. Blood glucose levels must be maintained within a narrow range (about 3.5-6.5 mM). Glucose levels lower than this range (hypoglycemia) may lead to mental confusion, coma, or death. High glucose levels (hyperglycemia) cause excessive thirst and frequent urination. Sustained hyperglycemia has been linked to several of complications of diabetes, including kidney damage, neural damage, and blindness.

Conventional glucose measurement techniques require lancing of a convenient part of the body (normally a fingertip) with a lancet, milking the finger to produce a drop of blood at the impalement site, and depositing the drop of blood on a measurement device (such as an analysis strip). This lancing method, at typical measurement frequencies of two to four times a day, is both painful and messy for the patient. The pain and inconvenience has additional and more serious implications of noncompliance. Patients generally avoid maintaining the recommended regimen of blood glucose measurement and thereby run the risk of improper glucose levels and consequent harmful effects.

Conditions worsen when there is a need for frequent blood glucose determination, such as when a diabetic patient is acutely ill, undergoing surgery, pregnant (or in childbirth), or suffering from severe ketoacidosis. Also, non-diabetic patients, such as the acutely ill patient treated with a pharmacologic dose of cbrticosteroid, or the patient with recurrent fainting spells who is suspected of having functional hypoglycemia, needs to have frequent serial blood glucose determinations made.

The conventional Point-of-Care (POC) techniques for diagnostic blood testing are routinely performed manually at the bedside using a small sample of blood. In addition, as mentioned above, home glucose monitoring by diabetics is also becoming increasingly routine in diabetes management. Patients are typically required to maintain logbooks for manually recording glucose readings and other relevant information. Even more specifically, patients now measure their blood glucose at scheduled times to determine the amount of insulin needed based on the current blood glucose result, and then record this information in a personal log book.

SureStep® Technology, developed by Lifescan, is one example of a conventional Point-of-Care home monitoring system. The SureStep® Technology, in one form, allows for single button testing, quick results, blood sample confirmation, and test memory. In operation, the SureStepe® Point-of-Care home monitoring system employs three steps. In a first step, the blood sample is applied to the test strip. The blood sample is deposited on a touchable absorbent pad. In addition, blood is retained and not transferred to other surfaces. The sample then flows one way through a porous pad to a reagent membrane, where a reaction occurs. The reagent membrane is employed to filter out red blood cells while allowing plasma to move through.

In a second step, the glucose reacts with reagents in the test strip. Glucose in the sample is oxidized by glucose oxidase (GO) in the presence of atmospheric oxygen, forming hydrogen peroxide (H₂O₂). H₂O₂ reacts with indicator dyes using horseradish peroxidase (HRP), forming a chromophore or light-absorbing dye. The intensity of color formed at the end of the reaction is proportional to the glucose present in the sample.

In a third step, the blood glucose concentration is measured with a meter. Reflectance photometry quantifies the intensity of the colored product generated by the enzymatic reaction. The colored product absorbs the light—the more glucose in a sample (and thus the more colored product on a test strip), the less reflected light. A detector captures the reflected light, converts it into an electronic signal, and translates it into a corresponding glucose concentration. The system is calibrated to yield plasma glucose values.

In addition, prior art devices have conventionally focused upon manually obtaining blood samples from in-dwelling catheters. Such catheters may be placed in venous or arterial vessels, centrally or peripherally. For example, Edwards LifeSciences' VAMP Plus Closed Blood Sampling System provides a safe method for the withdrawal of blood samples from pressure monitoring lines. The blood sampling system is designed for use with disposable and reusable pressure transducers and for connection to central line catheters, venous, and arterial catheters where the system can be flushed clear after sampling. The blood sampling system mentioned above, however, is for use only on patients requiring periodic withdrawal of blood samples from arterial and central line catheters that are attached to pressure monitoring lines.

The VAMP Plus design provides a closed and needleless blood sampling system, employing a blunt cannula for drawing of blood samples. In addition, a self-sealing port reduces the risk of infection by stopcock contamination. The VAMP Plus system employs a large reservoir with two sample sites. Two methods may be used to draw a blood sample in the VAMP Plus Closed Blood Sampling System. The first method, the syringe method for drawing blood samples, requires that the VAMP Plus is prepared for drawing a blood sample by drawing a clearing volume (preferred methods provided in the literature). To draw a blood sample, it is recommended that a preassembled packaged VAMP NeedleLess cannula and syringe is used. Then, the syringe plunger should be depressed to the bottom of the syringe barrel. The cannula is then pushed into the sampling site. The blood sample is drawn into the syringe. A blood transfer unit is employed to transfer the blood sample from the syringe to the vacuum tubes.

The second method allows for a direct draw of blood samples. Again, the VAMP Plus is first prepared for drawing a blood sample by drawing a clearing volume. To draw a blood sample, the VAMP Direct Draw Unit is employed. The cannula of the Direct Draw Unit is pushed into the sampling site. The selected vacuum tube is inserted into the open end of the Direct Draw Unit and the vacuum tube is filled to the desired volume.

The abovementioned prior art systems, however, have numerous disadvantages. In particular, manually obtaining blood samples from in-dwelling catheters tends to be cumbersome for the patient and healthcare providers. Moreover, it is impractical for the patient to use a bulky vacuum pump or power source as is suggested in the art.

The various elements of conventional blood parameter monitoring devices, and in particular, glucose monitoring devices, such as tubes, pumps, and lancets connecting the patient to the glucose meter unit tend to confine the patient and limit his mobility to various ambulatory locations. Additionally, the flexible tubing used by existing glucose meters is frequently damaged due to being wrapped around the glucose meter unit during its transport from one location to another.

Conventional Point-of-Care and home monitoring glucose meters also have substantial disadvantages. Since such portable meters can be used by a patient without a practitioner or supervisor, numerous errors can arise from these unsupervised procedures that may result in serious health risks for patients, which knowingly, or inadvertently, are not in compliance with medical directives. Additionally, patients often forget, or in some instances forego, conducting and correctly recording their glucose levels as measured by the instrument. If a patient skips a measurement they may even elect to write down a “likely” number in the notebook as if such a measurement had been taken.

In addition, physicians are subsequently faced with the task of carefully reviewing the hand-recorded data for use in optimizing the patient's diabetes therapy. In order to make intelligent and meaningful decisions regarding therapeutic modifications, it becomes necessary for the examining physician to not only summarize the available information but, more importantly, to analyze hundreds of time-dependant observations collected over an extended period of time in order to spot unusual and clinically significant features requiring any modifications of the patient's current diabetes management schedule. The recorded data typically extends over a period of time spanning several weeks or months and constitutes a vast amount of time-dependent data.

In the light of above described disadvantages, there is a need for improved methods and systems that can provide comprehensive blood parameter testing.

What is also needed is a programmable, automated system and method for obtaining blood samples for testing certain blood parameters and data management of measurement results, thus avoiding human recording errors and providing for central data analysis and monitoring.

### SUMMARY OF THE INVENTION

The present invention is directed towards an integrated, automated system for measurement and analysis of blood analytes and blood parameters. The present invention is also directed towards an automated blood parameter testing apparatus portion of the automated blood parameter analysis and measurement system. In one operation, system components are combined in a single apparatus and either programmed to initiate substantially automatic, periodic blood sampling or initiate substantially automatic blood sampling via operator input. The system operates substantially automatically to draw blood samples at suitable, programmable frequencies to analyze the drawn blood samples and obtain the desired blood readings such as glucose levels, hematocrit levels, hemoglobin blood oxygen saturation, blood gasses, lactates or any other parameter as would be evident to persons of ordinary skill in the art.

In one embodiment, the present invention is directed towards a substantially automated blood parameter testing apparatus in which one valve is employed. In another embodiment, the present invention is directed towards a substantially automated blood parameter testing apparatus that employs two valves. Optionally, the present invention is directed towards a substantially automated blood parameter testing apparatus in which a blood sensor is employed, in either the single valve or dual valve embodiment.

The present invention is also directed towards a substantially automated blood parameter testing apparatus that includes a plurality of sensors (such as single use sensors) that are packaged together in a cassette or cartridge (hereinafter, referred to as “sensor cassette”) for obtaining blood measurements. The sensors are preferably electrochemical or optochemical sensors, but other options such as sensors that support optical blood measurements (without relying on chemical reactions between the sample of blood and a chemical agent embedded in the sensor) are disclosed. The present invention is also directed towards apparatuses and methods that employ components of manual test systems (e.g. blood glucose test strips) for use in an automated measurement system.

The present invention is also directed towards an integrated, substantially automated blood parameter measurement and analysis system that employs a method of data transmission between the measuring device and portable monitors.

The present invention also advantageously measures a plurality of blood parameters and analytes, including, but not limited to glucose, hematocrit, heart rate, and hemoglobin oxygenation levels to improve the accuracy and reliability of the entire system.

In addition, the present invention is directed towards features of substantially automated blood analysis and measurement system, such as, but not limited to storage of measurement results for trending or later download and alerts or alarms based on predefined levels or ranges for blood parameters.

In one embodiment, the present invention is a device for substantially automatically obtaining a blood sample and determining the concentration of at least one analyte comprising a vascular access point; a tube originating from the vascular access point; a pump fixedly attached to the tube; a valve fixedly attached to the tube and located above the pump mechanism; at least one measurement element; a needleless port; and an electronic meter. In another embodiment, the device further comprises at least one capillary transport structure, preferably adapted to connect to the needleless port.

Preferably, the electronic meter of the present invention is a blood glucose monitor. Optionally, the blood contacting elements are disposable. In another embodiment, the blood contacting elements are contained in a disposable cartridge or cassette. Optionally, the disposable elements are mechanically, electrically or otherwise keyed to mate with reusable elements.

In one embodiment, the measurement element is a glucose oxidase test strip. Alternatively, the measurement is a sensor. Optionally, the sensor is contained in a sensor cassette. Still optionally, the sensor cassette comprises at least one pre-calibrated single use sensor. In addition, the sensor cassette may include a plurality of sensor cassettes, each comprising a different type of sensor.

In one embodiment, the sensor is an electrochemical sensor capable of detecting the presence of and enabling the measurement of the level of an analyte in a blood sample via electrochemical oxidation and reduction reactions at the sensor. In another embodiment, the sensor is an optochemical sensor capable of detecting the presence of and enabling the measurement of the level of an analyte in a blood or plasma.

In one embodiment, the present invention is a device for substantially automatically obtaining a blood sample and determining the concentration of at least one analyte comprising a vascular access point; a tube originating from the vascular access point; a pump mechanism fixedly attached to the tube; a valve fixedly attached to the tube and located above the pump mechanism; at least one measurement element; at least one capillary transport structure; a needleless port; and an electronic meter.

In another embodiment, the present invention is directed towards a method for automatically obtaining a blood sample and determining the concentration of at least one analyte comprising placing a vascular access point in a patient's blood vessel; closing a valve fixedly attached to the tube in response to a blood sample indication; creating suction in the tube by energizing a pump fixedly attached to the tube, with fluid contained in the tubing; withdrawing blood from the vascular access point of the patient; extending a capillary transport structure into a needleless port and filling said capillary transport with the blood withdrawn from the vascular access point of the patient; delivering the withdrawn blood to a measurement element, fixedly connected to the capillary transport structure; and calculating a blood parameter of the sample using an electronic meter.

In yet another embodiment the present invention is a device for substantially automatically obtaining a blood sample and determining the concentration of at least one analyte comprising a vascular access point; a tube originating from the vascular access point; a pump fixedly attached to the tube; a first valve fixedly attached to the tube and located above the pump; a second valve fixedly attached to said tube and located below the pump, wherein said second valve isolates the pump from vascular pressure; at least one measurement element; at least one capillary transport structure; a needleless port; and an electronic meter.

In another embodiment, the present invention is a method for automatically obtaining a blood sample and determining the concentration of at least one analyte comprising connecting a vascular access point of a patient to a tube; closing a first valve fixedly attached to the tube and located above the pump in response to a blood sample indication; creating suction in the tube by energizing a pump fixedly attached to the tube; withdrawing blood from the vascular access point of the patient; closing a second valve fixedly attached to the tube and located below the pump, wherein the second valve is used to isolate the reservoir from vascular pressure; extending a capillary transport member into a needleless port and filling said capillary transport with the blood withdrawn from the vascular access point of the patient; delivering the withdrawn blood to a measurement element, fixedly connected to said capillary transport structure; and calculating a blood parameter of the sample using an electronic meter.

In still another embodiment, the present invention is a device for automatically obtaining a blood sample and determining the concentration of at least one analyte comprising a vascular access point; a tube terminating at a vascular access point; a pump fixedly attached to the tube; a valve fixedly attached to the tube and located above the pump; at least one measurement element; at least one capillary transport structure; a needleless port; an electronic meter; and a sensor.

Optionally, the sensor is used for determining the presence of blood in the tube for analysis. Still optionally, the sensor is used for determining the presence of undiluted blood in the tube for analysis. And still optionally, the sensor is to verify that no bubbles are present in the fluid contained in the tube. In an alternative embodiment, the sensor is used to determine the oxygenation level of the blood and uses the oxygenation level to calibrate the glucose calculation. In yet another alternative embodiment, the sensor is used to determine the hemoglobin concentration and/or hematocrit of the blood and calibrates the glucose calculation.

In another embodiment, the present invention is a method for automatically obtaining a blood sample and determining the concentration of at least one analyte comprising connecting a vascular access point of a patient to a tube; closing a valve fixedly attached to the tube and located above the pump in response to a blood sample indication; creating suction in the tube by energizing a pump fixedly attached to the tube; withdrawing blood from the vascular access point of the patient; determining the presence of a blood sample via a blood sensor; extending a capillary transport member into a needle-less port and filling said capillary transport with the blood withdrawn from the vascular access point of the patient; delivering the withdrawn blood to a measurement element, fixedly connected to the capillary transport structure; and calculating a blood parameter of the sample using an electronic meter.

In yet another embodiment, the present invention is a device for automatically obtaining a blood sample and determining the concentration of at least one analyte comprising a vascular access point; a tube originating from the vascular access point; a pump fixedly attached to the tube; a first valve fixedly attached to the tube and located above the pump; a second valve fixedly attached to the tube and located below the pump; at least one measurement element; at least one capillary transport structure; a needle-less port; an electronic meter; and a blood sensor.

In still yet another embodiment, the present invention is a method for automatically obtaining a blood sample and determining the concentration of at least one analyte comprising connecting a vascular access point of a patient to a tube; closing a first valve fixedly attached to the tube and located above the pump in response to a blood sample indication; creating suction in the tube by energizing a pump fixedly attached to the tube; withdrawing blood from the vascular access point of the patient; determining the presence of a blood sample via a blood sensor; closing a second valve fixedly attached to the tube and located below the pump; extending a capillary transport member into a needle-less port and filling said capillary transport with the blood withdrawn from the vascular access point of the patient; delivering the withdrawn blood to a measurement element, fixedly connected to said capillary transport structure; and calculating a blood parameter of the sample using an electronic meter.

In one embodiment, the present invention is a system for automatically obtaining a blood sample and determining the concentration of at least one analyte comprising a monitor; a central monitoring station; and a blood parameter testing apparatus, further comprising: a vascular access point; a tube originating from the vascular access point; a pump fixedly attached to the tube; a valve fixedly attached to the tube and located above the pump mechanism; at least one measurement element; a needleless port; and an electronic meter.

In addition, the system of the present invention is in automatic operation and programmable to initiate a periodic sample reading. Optionally, the periodic sample reading is initiated via operator input. Preferably, data is transmitted between the blood parameter testing device and a monitor. Still preferably, the monitor maintains a record of at least one automated blood parameter testing device, at least one monitor, at least one patient, and at least one set of physiological parameters. Optionally, the measurement results are stored for trending or later download. Still optionally, the system alerts based on predefined levels or ranges for blood parameters.

In another embodiment, the present invention is a system for automatically obtaining a blood sample and determining the concentration of at least one analyte comprising a monitor; a central monitoring station; and a blood parameter testing apparatus, further comprising: a vascular access point; a tube originating from the vascular access point; a pump mechanism fixedly attached to the tube; a valve fixedly attached to the tube and located above the pump mechanism; at least one measurement element; at least one capillary transport structure; a needleless port; and an electronic meter.

In another embodiment, the present invention is a system for substantially automatically obtaining a blood sample and determining the concentration of at least one analyte comprising a monitor; a central monitoring station; and a blood parameter testing apparatus, further comprising: a vascular access point; a tube originating from the vascular access point; a pump fixedly attached to the tube; a first valve fixedly attached to the tube and located above the pump; a second valve fixedly attached to said tube and located below the pump, wherein said second valve isolates the pump from vascular pressure; at least one measurement element; at least one capillary transport structure; a needleless port; and an electronic meter.

In yet another embodiment, the present invention is a system for substantially automatically obtaining a blood sample and determining the concentration of at least one analyte comprising a monitor; a central monitoring station; and a blood parameter testing apparatus, further comprising: a vascular access point; a tube terminating at a vascular access point; a pump fixedly attached to the tube; a valve fixedly attached to the tube and located above the pump; at least one measurement element; at least one capillary transport structure; a needleless port; an electronic meter; and a sensor.

In still yet another embodiment, the present invention is a system for substantially automatically obtaining a blood sample and determining the concentration of at least one analyte comprising: a monitor; a central monitoring station; and a blood parameter testing apparatus, further comprising: a vascular access point; a tube originating from the vascular access point; a pump fixedly attached to the tube; a first valve fixedly attached to the tube and located above the pump; a second valve fixedly attached to the tube and located below the pump; at least one measurement element; at least one capillary transport structure; a needleless port; an electronic meter; and a blood sensor.

The aforementioned and other embodiments of the present invention shall be described in greater depth in the drawings and detailed description provided below.

### BRIEF DESCRIPTION OF THE DRAWINGS

These and other features and advantages of the present invention will be appreciated, as they become better understood by reference to the following Detailed Description when considered in connection with the accompanying drawings, wherein:

FIG. 1 depicts a block diagram of one use of the substantially automated blood parameter testing apparatus of the present invention, as employed in a substantially automated blood parameter measuring system;

FIG. 2a is a schematic diagram of one embodiment of the substantially automated blood parameter testing apparatus of the present invention;

FIG. 2b is a schematic diagram of one embodiment of the substantially automated blood parameter testing apparatus of the present invention;

FIG. 3a is a schematic diagram of one embodiment of the substantially automated blood parameter testing apparatus of the present invention;

FIG. 3b is a schematic diagram of one embodiment of the substantially automated blood parameter testing apparatus of the present invention;

FIG. 4 is a blood sensor as used in the circuit of the substantially automated blood parameter testing apparatus of the present invention;

FIG. 5 depicts a load cell on the plunger of the pump mechanism as used in the circuit of the substantially automated blood parameter testing apparatus of the present invention;

FIG. 6 illustrates components of the monitor of the substantially automated blood parameter analysis system of the present invention;

FIG. 7 depicts the components of a computing device as used in one embodiment of the substantially automated blood parameter analysis system of the present invention; and

FIG. 8 depicts communication channels between a plurality of monitors with a central monitoring station in the blood parameter analysis system of the present invention.

### DETAILED DESCRIPTION OF THE INVENTION

The present invention is directed towards an integrated, substantially automated system for measurement and analysis of blood analytes and blood parameters. The present invention is also directed towards a substantially automated blood parameter testing apparatus portion of the blood parameter analysis and measurement system. In operation, system components are combined in a single apparatus and either programmed to initiate substantially automatic, periodic blood sampling or initiate substantially automatic blood sampling via operator input. The system operates substantially automatically to draw blood samples at suitable, programmable frequencies to analyze the drawn blood samples and obtain the desired blood readings such as glucose levels, hematocrit levels, hemoglobin blood oxygen saturation, blood gasses, lactates or any other parameter as would be evident to persons of ordinary skill in the art.

In one embodiment, the present invention is directed towards a substantially automated blood parameter testing apparatus in which one valve is employed. In another embodiment, the present invention is directed towards a substantially automated blood parameter testing apparatus that employs two valves. Optionally, the present invention is directed towards a substantially automated blood parameter testing apparatus in which a blood sensor is employed, in either the single valve or dual valve embodiment.

The present invention is also directed towards a substantially automated blood parameter testing apparatus that includes a plurality of sensors (such as single use sensors) that are packaged together in a cassette or cartridge (hereinafter, referred to as “sensor cassette”) for obtaining blood measurements. The sensors are preferably electrochemical or optochemical sensors, but other options such as sensors that support optical blood measurements (without relying on chemical reactions between the sample of blood and a chemical agent embedded in the sensor) are disclosed. The present invention also discloses apparatuses and methods that employ components of manual test systems (e.g. blood glucose test strips) for use in an automated measurement system.

The present invention is also directed towards an integrated, substantially automated blood parameter measurement and analysis system that employs a method of data transmission between the measuring device and portable monitors.

The present invention also advantageously measures a plurality of blood parameters and analytes, including, but not limited to glucose, hematocrit, heart rate, and hemoglobin oxygenation levels to improve the accuracy and reliability of the entire system.

In addition, the present invention is directed towards features of the substantially automated blood analysis and measurement system, such as, but not limited to storage of measurement results for trending or later download and alerts or alarms based on predefined levels or ranges for blood parameters.

As referred to herein, the terms “blood analyte(s)” and “blood parameter(s)” refers to such measurements as, but not limited to, glucose level; ketone level; hemoglobin level; hematocrit level; lactate level; electrolyte level (Na⁺, K⁺, Cl⁻, Mg²⁺, Ca²⁺); blood gases (pO₂, pCO₂, pH); cholesterol; bilirubin level; and various other parameters that can be measured from blood or plasma samples. The term “vascular access point(s)” refer to venous or arterial access points in the peripheral or central vascular system.

In one embodiment, the integrated, substantially automated blood parameter analysis and measurement system comprises a substantially automated blood parameter testing apparatus for measuring blood glucose levels.

Reference will now be made in detail to specific embodiments of the invention. While the invention will be described in conjunction with specific embodiments, it is not intended to limit the invention to one embodiment. Thus, the present invention is not intended to be limited to the embodiments described, but is to be accorded the broadest scope consistent with the disclosure set forth herein.

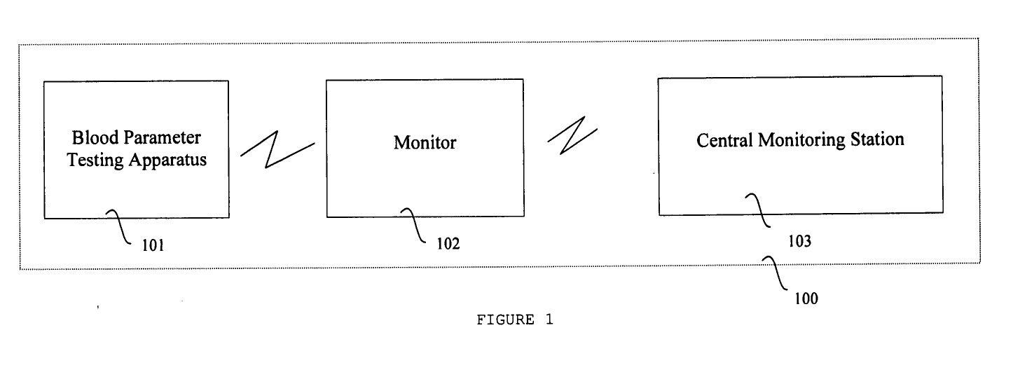

Referring to FIG. 1, a block diagram of one embodiment of the substantially automated blood parameter testing apparatus as used in a substantially automated blood parameter analysis and measurement system of the present invention is depicted. The system 100 comprises a substantially automated blood parameter testing apparatus 101, monitor 102, and a central monitoring station 103. In one embodiment, blood parameter testing apparatus 101 is employed to measure blood glucose levels. Blood parameter testing apparatus 101 is physically attached to a convenient part of the body, such as but not limited to, a fingertip of a patient (not shown) and is capable of providing monitor 102 with signals representing blood glucose data obtained from the patient. The glucose meter (not shown), is preferably portable and receives the blood sample, processes the contents of the blood, and calculates the glucose level in blood. The blood glucose level is displayed on the digital display of the glucose meter. In one embodiment, the processed data is transmitted to monitor 102. In yet another embodiment, the processed data is transmitted from monitor 102 to central monitoring station 103.

Central monitoring station 103 preferably maintains a record of all automated blood parameter testing apparatuses 101, monitors 102, patients (not shown), and physiological parameters measured over a period of time. In one embodiment, a plurality of monitors 102 communicates with a central monitoring station 103. Further, a plurality of substantially automated blood parameter testing apparatuses 101 communicates with one or more monitors 102.

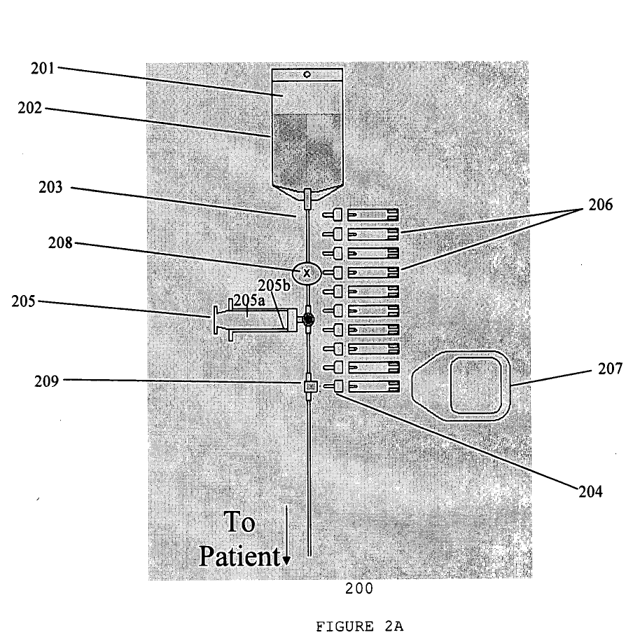

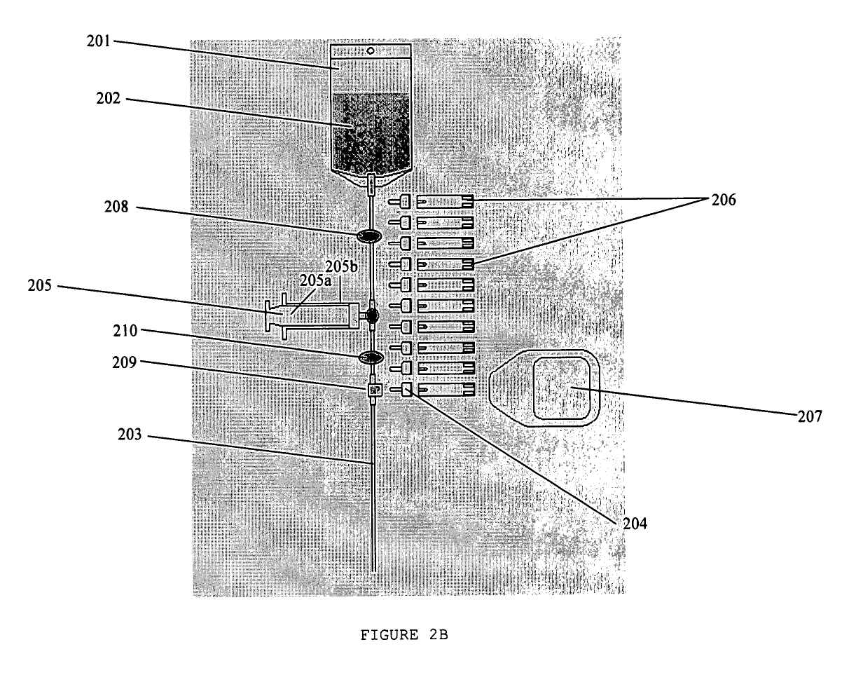

Referring to FIG. 2a, a schematic diagram depicts one embodiment of the substantially automated blood parameter testing apparatus of the present invention. As mentioned above, in one embodiment, the substantially automated blood parameter testing apparatus 200 tests for patient glucose levels. The substantially automated blood parameter testing apparatus 200 of the present invention comprises a “keep vein open” (hereinafter, “KVO”) reservoir 201 containing KVO solution 202, a distal tube 203 originating from the KVO reservoir 201 and terminating at a vascular access point (not shown) of the patient, and a pump mechanism 205 fixedly attached to tube 203.

Pump mechanism 205 is preferably a syringe, further comprising a plunger 205a and reservoir 205b, which are used to create suction or reverse pressure in the tube.

Substantially automated blood parameter testing apparatus 200 further comprises measurement element 206, preferably fixedly connected to and adaptable to connect to capillary transport structure 204. Preferably, the measurement chemistry is always mechanically isolated from the blood circuit.

Capillary transport structure 204 is adapted to connect to needle-less port 209. Needle-less port 209 is used to hold the sample of blood for measurement and analysis. Electronic meter 207 is used to check the blood glucose level. In one embodiment, electronic meter 207 is a standard point-of-contact glucose meter as are well-known to those of ordinary skill in the art.

The substantially automated blood parameter testing apparatus 200 of the present invention also comprises valve 208, fixedly attached to tube 203 and preferably located above pump mechanism 205.

In one embodiment, all blood contacting elements are disposable. In another embodiment, tube 203, capillary transport structure 204, pump mechanism 205, measurement element 206, valve 208 and needle-less port 209 are packaged as a disposable kit within a plastic package labeled for single patient use.

Alternatively, capillary transport structure 204 and measurement element 206 are packaged in a single sterile housing labeled for single patient use while tube 203, pump mechanism 205, valve 208, and needle-less port 209 are packaged in a separate, sterile housing labeled for single patient use. The elements in the two separate packages are removed from the packages and then combined by the end user at the time of use.

In a third embodiment, each individual combination of capillary transport structures 204 and measurement elements 206 are packaged in a separate, sterile compartment of a larger, multi-element package (hereinafter, referred to as a “cassette”) labeled for single patient use. The reusable mechanism automatically opens each individual sterile compartment at the time of use, thus acting as a dispenser.

In another embodiment, the disposable elements are mechanically, electrically, or otherwise keyed to mate with the reusable elements. Mechanical keys can take the form of a variety of three-dimensional, mating shapes, including, but not limited to cylinders, squares, or polygons of various configurations. Electrical keys can be of either analog or digital encoding schemes. Coding information may be transmitted by conventional electrical interfaces (connectors) or via short distance radiofrequency (RF) methods. Software keys may be in the form of a bar code or other passive encoding means. Coding information may be transmitted electrically, optically or by various means known to those skilled in the art.

In one embodiment, measurement element 206 is a glucose oxidase strip. In yet another embodiment, measurement element 206 is a sensor for performing blood analyte measurements, instead of a disposable test strip. In one embodiment, the sensor cassette is disposable and replaced periodically. The sensor cassette supports the use of at least one pre-calibrated single use sensor, and more preferably comprises a plurality of sensors arranged in a multiple layer tape structure. Each single-use sensor is advanced sequentially and positioned for direct contact with a blood sample through an advancement means.

The sensor employed is preferably an electrochemical sensor capable of detecting the presence of and enabling the measurement of the level of an analyte in a blood sample via electrochemical oxidation and reduction reactions at the sensor. Optionally, the sensor employed in the automated system for periodically measuring blood analytes and blood parameters is an optochemical sensor capable of detecting the presence of and enabling the measurement of the level of an analyte in a blood or plasma sample via optochemical oxidation and reduction reactions at the sensor. Optionally, the sensor cassette may include a plurality of sensor cassettes, each comprising a different type of sensor, capable of measuring a different blood parameter.

In another embodiment the sensor may optionally be a surface or miniature container, such as but not limited to a capillary tube, enabling storage of the blood sample for optical measurements. In this embodiment, both a light source and a light detector are used for measuring the blood analyte based on reflected, transmitted or other known optical effects such as Raman Spectroscopy, NIR or IR Spectroscopy, FTIR or fluoroscopy.

At rest, KVO solution 202 is in fluid communication with the vascular access point of the patient (not shown) and maintained at a slight positive pressure, usually by gravity. Thus, tube 203 is entirely filled with fluid. When a blood sample is indicated, either by a programmed response or operator indication, valve 208 is closed. Plunger 205a is extracted, filling reservoir 205b of pump mechanism 205 with fluid contained in the tubing and subsequently withdrawing blood from the vessel, by creating a negative pressure in tube 203. Capillary transport 204 is then extended into needle-less port 209 and is filled with blood. Capillary transport 204 is withdrawn from needle-less port 209 and delivers blood to measurement element 206. In one embodiment, measurement element 206 is a glucose oxidase strip. In an alternative embodiment, measurement element 206 is a sensor. Finally, electronic meter 207 calculates the glucose concentration of the blood sample.

Referring to FIG. 2b, a schematic diagram depicts a second embodiment of the substantially automated blood parameter testing apparatus of the present invention. In this particular embodiment, two valves are used to isolate the pump mechanism 205 from KVO and vascular pressure to manipulate the line. In other words, the actuator can be filled while controlling the pressure in the sample tubing.

As mentioned above, in one embodiment, substantially automated blood parameter testing apparatus 200 is a glucose meter. The automated blood parameter testing apparatus 200 of the present invention comprises a “keep vein open” (hereinafter, “KVO”) reservoir 201 containing KVO solution 202, a distal tube 203 originating from the KVO reservoir 201 and terminating at vascular access point of the patient (not shown), and pump mechanism 205 fixedly attached to tube 203.

Pump mechanism 205 is preferably a syringe, further comprising a plunger 205a and reservoir 205b, which are used to create suction or reverse pressure in the tube. However, it can include any other reverse pressure creating device.

Tube 203 further comprises a first valve 208 (upper valve) and second valve 210 (lower valve). First valve 208 controls the movement of the KVO solution 202 from reservoir 201 to the rest of the circuit. First valve 208 also monitors the rate of flow so that adjustments to the flow rate can be made appropriately.

In one embodiment, lower valve 210 is employed to isolate the pump mechanism 205 from KVO reservoir 202 and vascular pressure. As a result, lower valve 210 can help manipulate the pressure in the tube. Thus, by restricting both sides surrounding pump mechanism 205, it is possible to manipulate the pressure in the sample tube by moving the plunger 205a of pump mechanism 205 back and forth.

In one embodiment, substantially automated blood parameter testing apparatus 200 further comprises measurement element 206, preferably fixedly connected to and adaptable to connect to capillary transport structure 204. Preferably, the measurement chemistry is mechanically isolated from the blood circuit.

Capillary transport structure 204 is adapted to connect to needle-less port 209. Needle-less port 209 is used to hold the sample of blood for measurement and analysis. Electronic meter 207 is used to check the blood glucose level. In one embodiment, electronic meter 207 is a standard point-of-contact glucose meter as are well-known to those of ordinary skill in the art.

In one embodiment, measurement element 206 is a glucose oxidase strip. In yet another embodiment, measurement element 206 is a sensor for performing blood analyte measurements, instead of a disposable test strip. In one embodiment, the sensor cassette is disposable and replaced periodically. The sensor cassette supports the use of at least one pre-calibrated single use sensor, and more preferably comprises a plurality of sensors arranged in a multiple layer tape structure. Each single-use sensor is advanced sequentially and positioned for direct contact with a blood sample through an advancement means. The use of a sensor for the measurement has already been described with respect to FIG. 2a and thus will not be described in further detail herein.

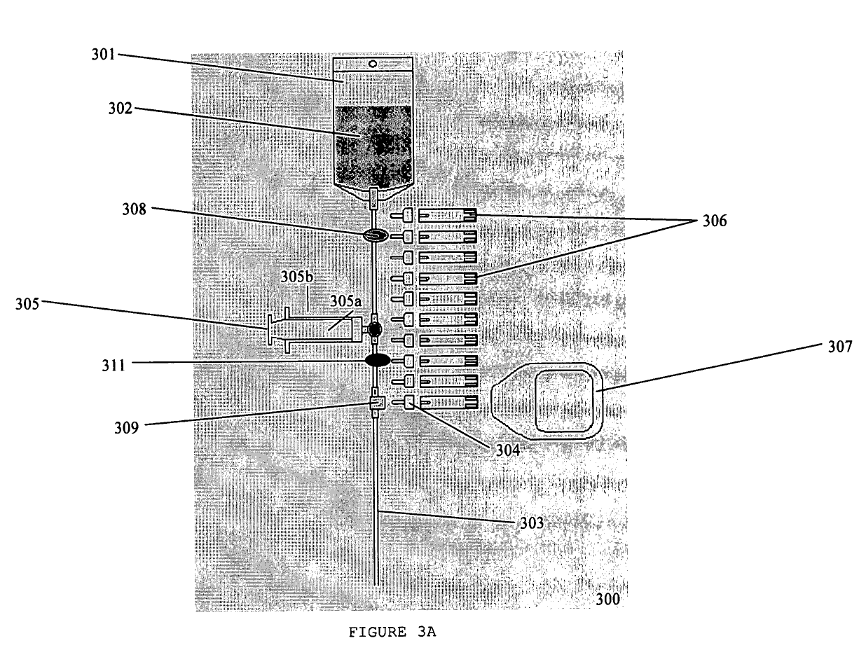

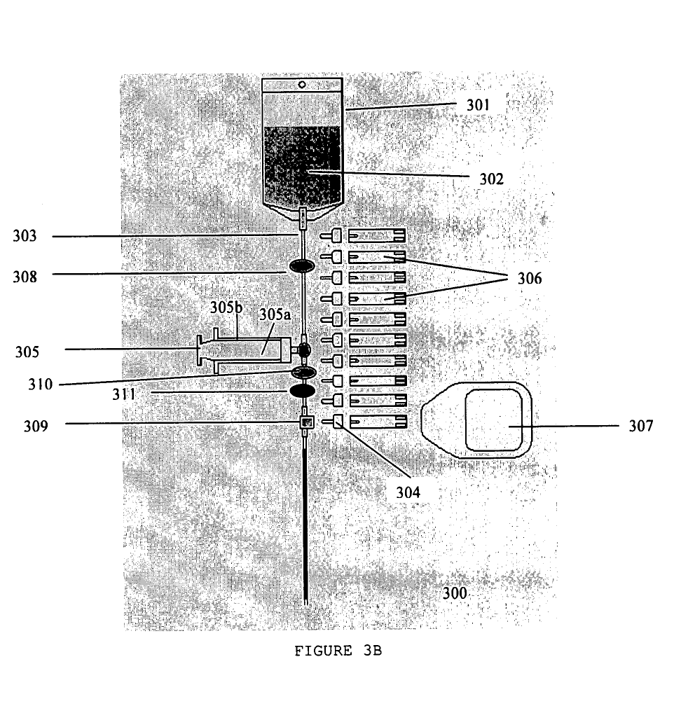

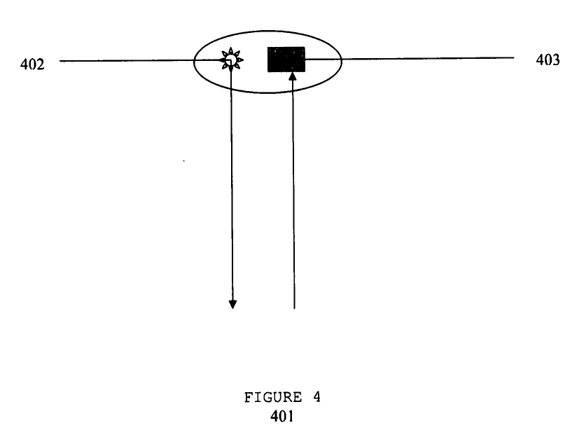

FIG. 3a is a schematic diagram depicting a third embodiment of the substantially automated blood parameter testing apparatus of the present invention. In this particular embodiment, one valve is used as in the first embodiment depicted in FIG. 2a however, a blood sensor (described in further detail below with respect to FIG. 4) is added to the circuit. The sensor is used for monitoring the presence or absence of blood in the circuit to enhance the reliability of the substantially automated blood parameter testing apparatus of the present invention. Although in a preferred embodiment the blood sensor is used for the detection of the presence of absence of blood in the circuit, it is not limited to such use. The sensor may be employed to detect the dilution of blood or detect other blood parameters, such as but not limited to, oxygenation, which are subsequently useful in improving the accuracy of the glucose determination.

Referring now to FIG. 3a, in one embodiment, the automated blood parameter testing apparatus 300 is a glucose meter. The substantially automated blood parameter testing apparatus 300 of the present invention comprises a “keep vein open” (hereinafter, “KVO”) reservoir 301 containing KVO solution 302, distal tube 303 originating from the KVO reservoir 301 and terminating at a vascular access point (not shown) of the patient, and a pump mechanism 305 fixedly attached to tube 303.

Pump mechanism 305 is preferably a syringe, further comprising a plunger 305a and reservoir 305b, which are used to create suction or reverse pressure in the tube.

Substantially automated blood parameter testing apparatus 300 further comprises measurement element 306, preferably fixedly connected to and adaptable to connect to capillary transport structure 304. The measurement chemistry is mechanically isolated from the blood circuit.

Capillary transport structure 304 is adapted to connect to needleless port 309. Needleless port 309 is used to hold the sample of blood for measurement and analysis. Electronic meter 307 is used to check the blood glucose level. In one embodiment, electronic meter 307 is a standard point-of-contact glucose meter as are well-known to those of ordinary skill in the art.

The substantially automated blood parameter testing apparatus 300 of the present invention also comprises valve 308, fixedly attached to tube 303 and preferably located above pump mechanism 305.

Substantially automated blood parameter testing apparatus 300 further comprises sensor 311, which is described in further detail below with respect to FIG. 4.

In one embodiment, measurement element 306 is a glucose oxidase strip. In yet another embodiment, measurement element 306 is a sensor for performing blood analyte measurements, instead of a disposable test strip. In one embodiment, the sensor cassette is disposable and replaced periodically. The sensor cassette supports the use of at least one pre-calibrated single use sensor, and more preferably comprises a plurality of sensors arranged in a multiple layer tape structure. Each single-use sensor is advanced sequentially and positioned for direct contact with a blood sample through an advancement means. The use of a sensor for the measurement has already been described with respect to FIG. 2a and thus will not be described in further detail herein.

In general, when the third embodiment of the blood parameter testing apparatus 300 of the present invention as described with respect to FIG. 3a above is in automatic operation, and the presence of a blood sample is indicated via blood sensor 311, valve 308 is closed and plunger 305a of pump mechanism 305 is extracted simultaneously. The vacuum or negative pressure created in tube 303 causes the blood in the blood vessel to rise up. The capillary transport 304 is then extended into needle-less port 309, which is subsequently filled with blood.

The blood sample collected in capillary transport 309 is used for the blood glucose measurement. In one embodiment, measurement element 306 is a glucose oxidase strip. After the blood sensor 311 confirms the presence of undiluted blood in the tube, the blood sensor 311 initiates a blood glucose measurement. In one embodiment, where glucose oxidase strips are employed instead of a measurement sensor, the glucose oxidase strip holder (not clearly shown) advances the next measurement element 306, which in this embodiment is a clean test strip. The advanced glucose oxidase test strip from the test strip holder then reaches needleless port 309 electromechanically, wherein a sample of blood (usually a drop) is placed on the test strip. The glucose oxidase test strip is then inserted into electronic meter 307, which then performs the blood analysis.

The blood sample on the reagent strip reacts with the reagents in the reagent strip; thus, the resulting color change is read from the back side of the test strip via the optical sensor. The optical sensor signals are converted by electronic meter 307 into a numerical readout on display, which reflects a numerical glucose level of the blood sample.

Referring to FIG. 3b, a schematic diagram depicts another embodiment of the substantially automated blood parameter testing apparatus of the present invention. In this particular embodiment, two valves are employed for fluid control, as in FIG. 2b however a blood sensor is added to the circuit. Two valves are used to isolate the pump mechanism 305 from any KVO and vascular pressure to manipulate the line. The actuator can thus be fired while controlling the pressure in the sample tubing.

As shown in FIG. 3b, as mentioned above, in one embodiment, the substantially automated blood parameter testing apparatus 300 is a glucose meter. The substantially automated blood parameter testing apparatus 300 of the present invention comprises a “keep vein open” (hereinafter, “KVO”) reservoir 301 containing KVO solution 302, distal tube 303 originating from the KVO reservoir 301 and terminating at a vascular access point of the patient (not shown), and pump mechanism 305 fixedly attached to tube 303. Tube 303 further comprises a first valve 308 (upper valve) and second valve 310 (lower valve). First valve 308 controls the movement of the KVO solution 302 from reservoir 301 to the rest of the circuit. First valve 308 also monitors the rate of flow so that adjustments to the flow rate can be made appropriately.

In one embodiment, lower valve 310 is employed to isolate the pump mechanism 305 from KVO reservoir 302 and vascular pressure. As a result, lower valve 310 can help manipulate the pressure in the tube. Thus, by restricting both sides of the tube surrounding pump mechanism 305, it is possible to manipulate the pressure in the sample tube by moving the plunger 305a of pump mechanism 305 back and forth.

Pump mechanism 305 is preferably a syringe, further comprising a plunger 305a and reservoir 305b, which are used to create suction or reverse pressure in the tube.

Substantially automated blood parameter testing apparatus 300 further comprises measurement element 306, preferably fixedly connected to and adaptable to connect to capillary transport structure 304. The measurement chemistry is mechanically isolated from the blood circuit.

Capillary transport structure 304 is adapted to connect to needleless port 309. Needleless port 309 is used to hold the sample of blood for measurement and analysis. Electronic meter 307 is used to check the blood glucose level. In one embodiment, electronic meter 307 is a standard point-of-contact glucose meter as are well-known to those of ordinary skill in the art.

The substantially automated blood parameter testing apparatus 300 of the present invention also comprises valve 308, fixedly attached to tube 303 and preferably located above pump mechanism 305.

Substantially automated blood parameter testing apparatus 300 further comprises sensor 311, which is described in further detail below with respect to FIG. 4.

In one embodiment, measurement element 306 is a glucose oxidase strip. In yet another embodiment, measurement element 306 is a sensor for performing blood analyte measurements, instead of a disposable test strip. In one embodiment, the sensor cassette is disposable and replaced periodically. The sensor cassette supports the use of at least one pre-calibrated single use sensor, and more preferably comprises a plurality of sensors arranged in a multiple layer tape structure. Each single-use sensor is advanced sequentially and positioned for direct contact with a blood sample through an advancement means. The use of a sensor for the measurement has already been described with respect to FIG. 2a and thus will not be described in further detail herein.

In one embodiment, the blood parameter testing apparatus is in automatic operation. The automated device is programmable to initiate a sample reading periodically or via operator input. Operator input is initiated by, but not limited to, the push of a button. Once a button is pushed, control signals are sent to the aforementioned operational components to obtain a blood sample, sample the blood, and measure blood analytes. In addition, operator input may be initiated at the central monitoring station.

Referring now to FIG. 4, a blood sensor as used in the circuit of the substantially automated blood parameter testing apparatus of the present invention is depicted. The sensor is used for monitoring the presence or absence of blood in the circuit to enhance the reliability of the substantially automated blood parameter testing apparatus of the present invention. Although in an embodiment the blood sensor is used for the detection of the presence of absence of blood in the circuit, it is not limited to such use. The sensor may be employed to detect the dilution of blood or detect other blood parameters, such as but not limited to, oxygenation, which are subsequently useful in improving the accuracy of the glucose determination.

Blood sensor 401 comprises an illumination source 402 and a detector 403. Illumination source 402 is used to trans-illuminate the tubing. The illumination source can be a single, multi-wavelength laser diode, a tunable laser or a series of discrete LEDs or laser diode elements, each emitting a distinct wavelength of light selected from the near infrared region. Alternatively, the illumination source can be a broadband near infrared (IR) emitter, emitting wavelengths as part of a broadband interrogation burst of IR light or radiation, such as lamps used for spectroscopy.

At least one detector 403 detects light reflected and/or transmitted by sample blood. The wavelength selection can be performed by either sequencing single wavelength light sources or by wavelength selective elements, such as using different filters for the different detectors or using a grating that directs the different wavelengths to the different detectors. The detector array converts the reflected light into electrical signals indicative of the degree of absorption light at each wavelength and transfers the converted signals to an absorption ratio analyzer such as a microprocessor. The analyzer processes the electrical signals and derives an absorption (e.g., a reflection and/or transmittance) ratio for at least two of the wavelengths. The analyzer then compares the calculated ratio with predetermined values to detect the concentration and/or presence of an analyte such as, but not limited to glucose, hematocrit levels and/or hemoglobin oxygenation levels in the patient blood sample. For example, changes in the ratios can be correlated with the specific near infrared (IR) absorption peak for glucose at about 1650 nm or 2000-2500 nm or around 10 micron, which varies with concentration of the blood analyte.

In one embodiment, blood sensor 401 establishes the presence of blood in the tube and subsequently activates other components of the blood parameter testing apparatus, such as advancement of a glucose oxidase strip and measurement by the electronic meter, for further analysis of the blood sample. Blood sensor 401 also determines whether the blood available in the tube is undiluted and bubble-free in the fluid circuit.

As described above, the method of detecting whether undiluted blood has reached the proximity of the sensor and is ready for sampling is to illuminate the tubing in the proximity of the sensor. Based upon the transmitted and/or reflected signal, the device can establish whether the fluid in the specific segment is undiluted blood. Dead space is managed by actively sensing the arrival and departure of blood within the disposable sensor cassette.

In addition, blood sensor 401 is capable of other blood analysis functions, including but not limited to, determining the oxygenation level of the blood and using the oxygen status to adjust or calibrate the glucose calculation. In an exemplary embodiment, the optically measured hematocrit level is used to correct for the influence of hemodilution on blood analytes such as, but not limited to, glucose. Preferably, hematocrit levels and hemoglobin oxygenation levels are accurately measured using two or more wavelengths. If the hematocrit level is high or low it may alter the results, owing to factors that are separate from yet compounded by the effects of different water distribution in the different blood components. The glucose reading is thus more accurate when the hemoglobin oxygenation and hematocrit levels are taken into account. Other combinations regarding the number and type of optical wavelengths and the parameters to be corrected can be used according to known correlations between blood parameters.

In another embodiment, the optical sensor is configured for measuring glucose directly and repeatably, replacing the single use chemistry strips and blood sampling mechanism completely.

In yet another embodiment, a reusable electrode is brought into fluid contact with the circuit, replacing the single use chemistry and blood sampling mechanism completely. Optionally, the reusable electrode replaces the single use chemistry strips, but not the blood sampling mechanism.

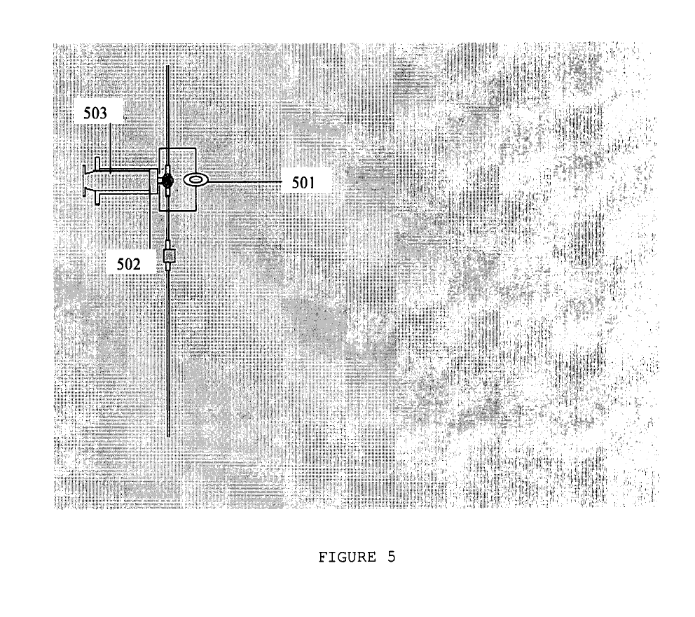

Referring now to FIG. 5, a load cell on the plunger of the pump mechanism of the abovementioned circuit of the present invention is depicted. In one embodiment, in order to measure and manipulate the pressure within the tube, load cell 501 can be retrofitted on pump mechanism (syringe) 503. By pinching both the sides of the tube and moving plunger 502 forward and backward it is possible to manipulate the pressure in the sample tube. Load cell 501 with a digital readout capability measures the force on the plunger 502 and can thus be adjusted. Due to the efficient control of the plunger via the load cell, and subsequent efficient pressure management in the tubing, the amount of blood required for a sample is minimized.

In another embodiment, the pressure inside the tubing is monitored directly by a conventional, discrete pressure transducer.

As described with reference to FIG. 1 above, in one embodiment, the blood parameter testing apparatus of the present invention is set up to communicate with patient monitors and/or central stations and/or the internet. Once the blood glucose level of the patient is ascertained, the processed data from the glucose meter is stored in the local memory of the glucose meter and subsequently transmitted to a monitor. In one embodiment, the data stored within the glucose meter is preferably transferred to the monitor through appropriate communication links and an associated data modem. In an alternative embodiment, data stored within the glucose meter may be directly downloaded into the monitor through an appropriate interface cable.

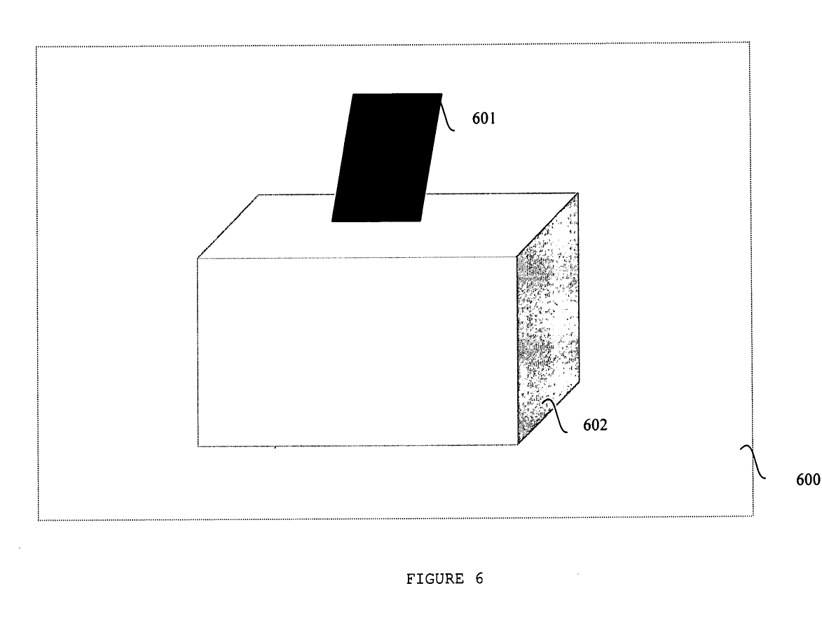

Referring now to FIG. 6, the components of the monitor as used in the substantially automated blood parameter measuring system of the present invention is depicted. Monitor 600 comprises a glucose meter card 601 and a computing device 602, which are preferably portable. Computing device 602 may be, but is not limited to, a portable computer such as personal digital assistant (PDAs), electronic notebook, pager, watch, cellular telephone and electronic organizer. Glucose meter card 601 is connected to or docked with computing device 602 to form an integral unit. Glucose meter card 601 may be inserted into an access slot (not shown) in computing device 602, may grip its housing, or interconnect in any other suitable manner as is well known to those of skill in the art. When glucose meter card 601 is docked with computing device 602, computing device 602 identifies the card 601 and loads the required software either from its own memory or from the card.

Thus, glucose meter card 601 includes the software necessary to process, analyze and interpret the recorded diabetes patient data and generate an appropriate data interpretation output. The results of the data analysis and interpretation performed upon the stored patient data by the monitor 600 are displayed in the form of a paper report generated through a printer (not shown) associated with the monitor 600. Alternatively, the results of the data interpretation procedure may be directly displayed on a graphical user interface unit (not shown) associated with the central monitoring station (not shown).

The software uses a blend of symbolic and numerical methods to analyze the data, detect clinical implications contained in the data and present the pertinent information in the form of a graphics-based data interpretation report. The symbolic methods used by the software encode the logical methodology used by expert diabetologists as they examine patient logs for clinically significant findings, while the numeric or statistical methods test the patient data for evidence to support a hypothesis posited by the symbolic methods which may be of assistance to a reviewing physician.

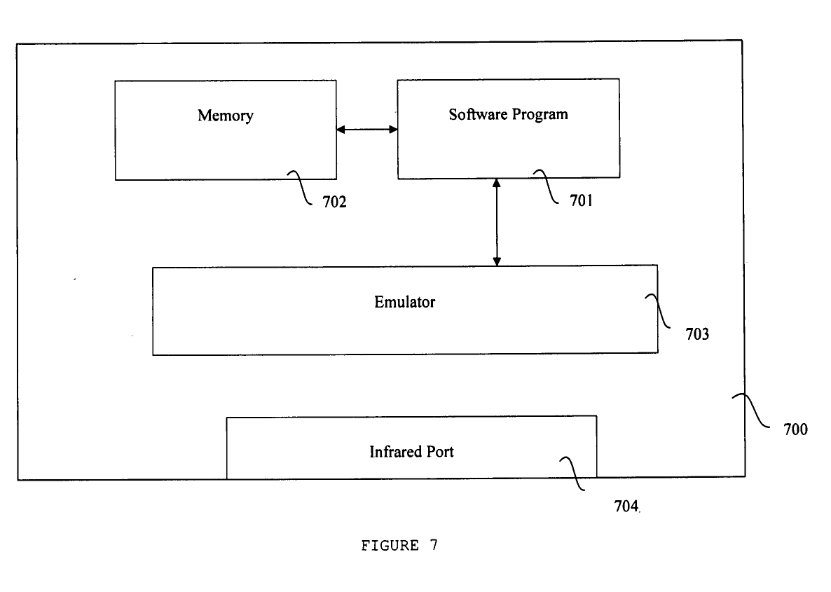

Referring to FIG. 7, the diagram depicts the components of a computing device as used in the blood parameter analysis system of the present invention. Computing device 700 preferably comprises software program 701, memory 702, emulator 703, and infrared port 704. Upon user request the information from the central monitoring station is received by software program 701 and stored in memory 702.

Software program 701 allows the user to perform queries on the stored information. For example, the user may wish to view a selected group of patients or all patients under observation. The user may set an alarm, when a desired sensor is in operation. The results of the user's query are displayed through a graphical user interface (GUI) on a display panel (not shown).

Operationally, a user may choose a person to be examined by selecting the appropriate glucose meter unit attached to that individual, using the GUI application. Each glucose meter consists of a unique identification. The selection causes the emulator, which emulates a remote control, to send instructions for that particular glucose meter. The instructions are sent via an infrared signal transmitted from the infrared port of the monitor to the photodetector (not shown) of the glucose meter, which is further conveyed to the sensor unit. The sensor unit is now initiated to communicate with the monitor. The monitor then receives the physiological signals from sensor unit and measures the desired physiological parameter.

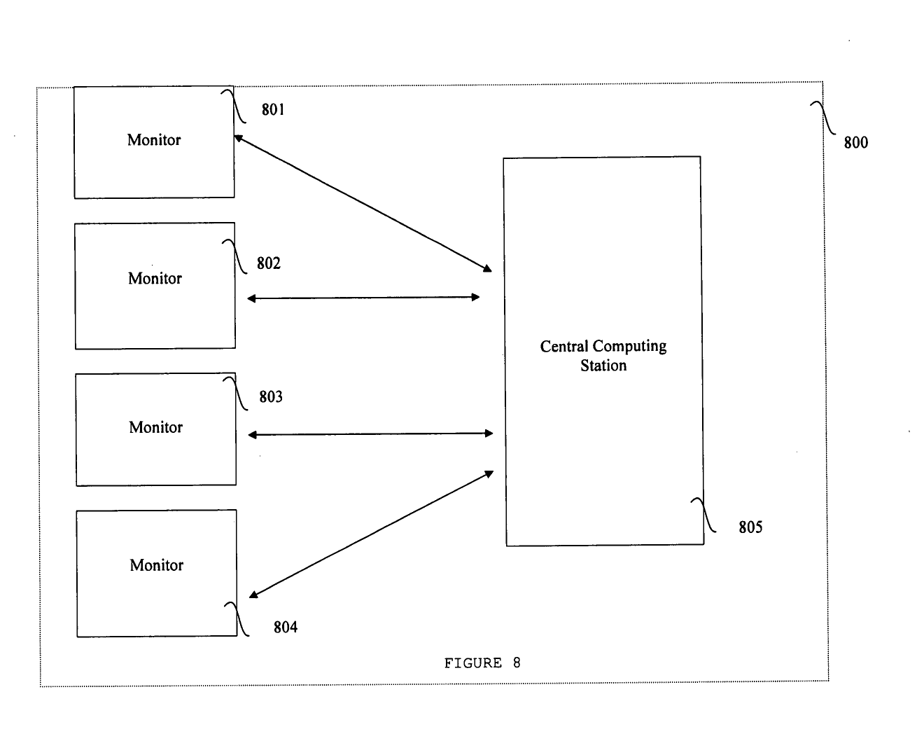

Referring now to FIG. 8, the diagram depicts a communication scheme between plurality of monitors 801, 802, 803, and 804 with central monitoring station 805. Monitors 801, 802, 803, and 804 wirelessly transmit vital patient information, including but not limited to the measured blood glucose level to central monitoring station 805. Medical conditions of a plurality of individual patients can be monitored from central monitoring station 805. An online database of the patients can be easily transported using a suitable relational database management system and an appropriate application programming language to the web server to make patient health conditions available on the World Wide Web.

In an alternative embodiment, either single or multiple lumen tubing structures may be attached to the catheter attached to the vascular access point. The tubing structure may vary depending upon functional and structural requirements of the system and are not limited to the embodiments described herein.

The substantially automated system for periodically measuring blood analytes and blood parameters further includes alerts and integrated test systems. The alerts may include alerts for detection of air in a line and detection of a blocked tube. In addition, the alerts may include alerts for hyperglycemia and hypoglycemia. The alerts may also include alerts for a hemoglobin level below a defined level.

Optionally, the control unit of the automated system for periodically measuring blood analytes and blood parameters enables input of user-defined ranges for blood parameters. Still optionally, the system alerts the user when the blood measurement falls outside of the user-defined ranges for blood parameters. Still optionally, the data from the system is correlated with other blood parameters to indicate an overall patient condition.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention is not to be limited to the details given herein, but may be modified within the scope of the appended claims.

We claim:

1. A device for substantially automatically obtaining a blood sample and determining the concentration of at least one analyte comprising:
a vascular access point;
a tube originating from the vascular access point;
a pump fixedly attached to the tube;
a valve fixedly attached to the tube and located above the pump mechanism;
at least one measurement element;
a needleless port; and
an electronic meter.

2. The device of claim 1 further comprising at least one capillary transport structure.

3. The device of claim 2 wherein the at least one capillary transport structure is adapted to connect to the needle-less port.

4. The device of claim 1 wherein the pump is a syringe, further comprising a plunger and a reservoir.

5. The device of claim 1 wherein the electronic meter is a blood glucose monitor.

6. The device of claim 1 wherein blood contacting elements are disposable.

7. The device of claim 6 wherein blood contacting elements are contained in a disposable cartridge or cassette.

8. The device of claim 6 wherein the disposable elements are mechanically, electrically or otherwise keyed to mate with reusable elements.

9. The device of claim 1 wherein the chemistry is mechanically isolated from the blood circuit.

10. The device of claim 1 wherein the measurement element is a glucose oxidase test strip.

11. The device of claim 1 wherein the measurement element is a sensor.

12. The device of claim 11 wherein the sensor is contained in a sensor cassette.

13. The device of claim 12 wherein the sensor cassette is disposable.

14. The device of claim 12 wherein the sensor cassette comprises at least one pre-calibrated single use sensor.

15. The device of claim 12 wherein the sensor cassette comprises a plurality of sensors arranged in a multiple layer tape structure.

16. The device of claim 14 wherein each single-use sensor is advanced sequentially and positioned for direct contact with a blood sample through an advancement means.

17. The device of claim 11 wherein the sensor cassette includes a plurality of sensor cassettes, each comprising a different type of sensor.

18. The device of claim 11 wherein the sensor is an electrochemical sensor capable of detecting the presence of and enabling the measurement of the level of an analyte in a blood sample via electrochemical oxidation and reduction reactions at the sensor.

19. The device of claim 11 wherein the sensor is an optochemical sensor capable of detecting the presence of and enabling the measurement of the level of an analyte in a blood or plasma.

20. The device of claim 11 wherein the sensor determines the oxygenation level of the blood and uses the oxygenation level to calibrate the glucose calculation.

21. The device of claim 11 wherein the sensor determines the hemoglobin concentration and/or hematocrit of the blood and calibrates the glucose calculation.

22. The device of claim 1 wherein the needleless port is used to hold the sample of blood for glucose measurement.

23. The device of claim 1 wherein the blood sample is obtained at predetermined, programmable time intervals or operator indication.

24. The device of claim 23 wherein the operator indication is via a push button.

25. A device for substantially automatically obtaining a blood sample and determining the concentration of at least one analyte comprising:
a vascular access point;
a tube originating from the vascular access point;
a pump mechanism fixedly attached to the tube;
a valve fixedly attached to the tube such that said pump lies between said valve and the vascular access point;
at least one measurement element;
at least one capillary transport structure;
a needleless port; and
an electronic meter.

26. The device of claim 25 wherein the at least one capillary transport structure is adapted to connect to the needleless port.

27. The device of claim 25 wherein the pump is a syringe, further comprising a plunger and a reservoir.

28. The device of claim 25 wherein the electronic meter is a blood glucose monitor.

29. The device of claim 25 wherein blood contacting elements are disposable.

30. The device of claim 29 wherein blood contacting elements are contained in a disposable cartridge or cassette.

31. The device of claim 29 wherein the disposable elements are mechanically, electrically or otherwise keyed to mate with reusable elements.

32. The device of claim 25 wherein the chemistry is mechanically isolated from the blood circuit.

33. The device of claim 25 wherein the measurement element is a glucose oxidase test strip.

34. The device of claim 25 wherein the measurement element is a sensor.

35. The device of claim 34 wherein the sensor is contained in a sensor cassette.

36. The device of claim 35 wherein the sensor cassette is disposable.

37. The device of claim 35 wherein the sensor cassette comprises at least one pre-calibrated single use sensor.

38. The device of claim 35 wherein the sensor cassette comprises a plurality of sensors arranged in a multiple layer tape structure.

39. The device of claim 37 wherein each single-use sensor is advanced sequentially and positioned for direct contact with a blood sample through an advancement means.

40. The device of claim 35 wherein the sensor cassette includes a plurality of sensor cassettes, each comprising a different type of sensor.

41. The device of claim 35 wherein the sensor is an electrochemical sensor capable of detecting the presence of and enabling the measurement of the level of an analyte in a blood sample via electrochemical oxidation and reduction reactions at the sensor.

42. The device of claim 35 wherein the sensor is an optochemical sensor capable of detecting the presence of and enabling the measurement of the level of an analyte in a blood or plasma sample via optochemical oxidation and reduction reactions at the sensor.

43. The device of claim 35 wherein the sensor determines the oxygenation level of the blood and uses the oxygenation level to calibrate the glucose calculation.

44. The device of claim 35 wherein the sensor determines the hemoglobin concentration and/or hematocrit of the blood and calibrates the glucose calculation.

45. The device of claim 25 wherein the needle-less port is used to hold the sample of blood for glucose measurement.

46. The device of claim 25 wherein the blood sample is obtained at predetermined, programmable time intervals or operator indication.

47. The device of claim 47 wherein the operator indication is via a push button.

48. A method for substantially automatically obtaining a blood sample and determining the concentration of at least one analyte comprising:
creating a vascular access point in association with a patient's blood vessel;
attaching a tube to said vascular access point;
closing a valve fixedly attached to the tube in response to a blood sample indication;
creating suction in the tube by activating a pump fixedly attached to the tube, with fluid contained in the tubing;
withdrawing blood from the vascular access point of the patient;
extending a capillary transport structure into a needleless port and filling said capillary transport with the blood withdrawn from the vascular access point of the patient;
delivering the withdrawn blood to a measurement element, fixedly connected to the capillary transport structure; and
calculating a blood parameter of the sample using an electronic meter.

49. The method of claim 48 wherein the at least one capillary transport structure is adapted to connect to the needleless port.

50. The method of claim 48 wherein the pump is a syringe, further comprising a plunger and a reservoir.

51. The method of claim 48 wherein the electronic meter is a blood glucose monitor.

52. The method of claim 48 wherein blood contacting elements are disposable.

53. The method of claim 52 wherein blood contacting elements are contained in a disposable cartridge or cassette.

54. The method of claim 52 wherein the disposable elements are mechanically, electrically or otherwise keyed to mate with reusable elements.

55. The method of claim 48 wherein the chemistry is mechanically isolated from the blood circuit.

56. The method of claim 48 wherein the measurement element is a glucose oxidase test strip.

57. The method of claim 48 wherein the measurement element is a sensor.

58. The method of claim 57 wherein the sensor is contained in a sensor cassette.

59. The method of claim 58 wherein the sensor cassette is disposable.

60. The method of claim 58 wherein the sensor cassette comprises at least one pre-calibrated single use sensor.

61. The method of claim 58 wherein the sensor cassette comprises a plurality of sensors arranged in a multiple layer tape structure.

62. The method of claim 60 wherein each single-use sensor is advanced sequentially and positioned for direct contact with a blood sample through an advancement means.

63. The method of claim 58 wherein the sensor cassette includes a plurality of sensor cassettes, each comprising a different type of sensor.

64. The method of claim 57 wherein the sensor is an electrochemical sensor capable of detecting the presence of and enabling the measurement of the level of an analyte in a blood sample via electrochemical oxidation and reduction reactions at the sensor.

65. The method of claim 57 wherein the sensor is an optochemical sensor capable of detecting the presence of and enabling the measurement of the level of an analyte in a blood or plasma sample.

66. The method of claim 57 wherein the sensor determines the oxygenation level of the blood and uses the oxygenation level to calibrate the glucose calculation.

67. The method of claim 57 wherein the sensor determines the hemoglobin concentration and/or hematocrit of the blood and calibrates the glucose calculation.

68. The method of claim 48 wherein the needleless port is used to hold the sample of blood for glucose measurement.

69. The method of claim 48 wherein the blood sample is obtained at predetermined, programmable time intervals or operator indication.

70. The method of claim 69 wherein the operator indication is via a push button.

71. A device for substantially automatically obtaining a blood sample and determining the concentration of at least one analyte comprising:
a vascular access point;
a tube originating from the vascular access point;
a pump fixedly attached to the tube;
a first valve fixedly attached to the tube such that said pump lies between the first valve and vascular access point;
a second valve fixedly attached to said tube such that said second valve lies between the pump and the vascular access point, wherein said second valve isolates the pump from vascular pressure;
at least one measurement element;
at least one capillary transport structure;
a needleless port; and
an electronic meter.

72. A method for substantially automatically obtaining a blood sample and determining the concentration of at least one analyte comprising:
connecting a vascular access point of a patient to a tube;
closing a first valve fixedly attached to the tube and located above the pump in response to a blood sample indication;
creating suction in the tube by energizing a pump fixedly attached to the tube;
withdrawing blood from the vascular access point of the patient;
closing a second valve fixedly attached to the tube and located below the pump, wherein the second valve is used to isolate the reservoir from vascular pressure;
extending a capillary transport member into a needleless port and filling said capillary transport with the blood withdrawn from the vascular access point of the patient;
delivering the withdrawn blood to a measurement element, fixedly connected to said capillary transport structure; and
calculating a blood parameter of the sample using an electronic meter.

73. A device for substantially automatically obtaining a blood sample and determining the concentration of at least one analyte comprising:
a tube terminating at a vascular access point;
a pump fixedly attached to the tube;
a valve fixedly attached to the tube and located above the pump;
at least one measurement element;
at least one capillary transport structure;
a needleless port;
an electronic meter; and
a sensor.

74. The device of claim 73 wherein said sensor is used for determining the presence of blood in the tube for analysis.

75. The device of claim 73 wherein said sensor is used for determining the presence of undiluted blood in the tube for analysis.

76. The device of claim 73 wherein said sensor is used for verifying that no bubbles are present in the fluid contained in the tube.

77. The device of claim 73 wherein the sensor determines the oxygenation level of the blood and uses the oxygenation level to calibrate the glucose calculation.

78. The device of claim 73 wherein the sensor determines the hemoglobin concentration and/or hematocrit of the blood and calibrates the glucose calculation.

79. A method for substantially automatically obtaining a blood sample and determining the concentration of at least one analyte comprising:
connecting a vascular access point of a patient to a tube;
closing a valve fixedly attached to the tube and located above the pump in response to a blood sample indication;
creating suction in the tube by energizing a pump fixedly attached to the tube;
withdrawing blood from the vascular access point of the patient;
determining the presence of a blood sample via a blood sensor;
extending a capillary transport member into a needle-less port and filling said capillary transport with the blood withdrawn from the vascular access point of the patient;
delivering the withdrawn blood to a measurement element, fixedly connected to the capillary transport structure; and
calculating a blood parameter of the sample using an electronic meter.

80. A device for substantially automatically obtaining a blood sample and determining the concentration of at least one analyte comprising:
a tube originating from a vascular access point;
a pump fixedly attached to the tube;
a first valve fixedly attached to the tube and located above the pump;
a second valve fixedly attached to the tube and located below the pump;
at least one measurement element;
at least one capillary transport structure;
a needle-less port;
an electronic meter; and
a blood sensor.

81. A method for substantially automatically obtaining a blood sample and determining the concentration of at least one analyte comprising:
connecting a vascular access point of a patient to a tube;
closing a first valve fixedly attached to the tube and located above the pump in response to a blood sample indication;
creating suction in the tube by activating a pump fixedly attached to the tube;
withdrawing blood from the vascular access point of the patient;
determining the presence of a blood sample via a blood sensor;
closing a second valve fixedly attached to the tube and located below the pump;
extending a capillary transport member into a needle-less port and filling said capillary transport with the blood withdrawn from the vascular access point of the patient;
delivering the withdrawn blood to a measurement element, fixedly connected to said capillary transport structure; and
calculating a blood parameter of the sample using an electronic meter.

82. A system for substantially automatically obtaining a blood sample and determining the concentration of at least one analyte comprising:
a monitor;
a central monitoring station; and
a blood parameter testing apparatus, further comprising:
a vascular access point;
a tube originating from the vascular access point;
a pump fixedly attached to the tube;
a valve fixedly attached to the tube and located above the pump mechanism;
at least one measurement element;
a needleless port; and
an electronic meter.

83. The system of claim 82 wherein the blood parameter testing apparatus further comprises at least one capillary transport structure.

84. The system of claim 82 wherein the blood parameter testing device is a blood glucose monitor.

85. The system of claim 82 wherein the blood parameter testing device is in automatic operation.

86. The system of claim 82 wherein the automatic blood parameter testing device is programmable to initiate a periodic sample reading.

87. The system of claim 82 wherein a periodic sample reading is initiated via operator input.

88. The system of claim 82 wherein data is transmitted between the blood parameter testing device and a monitor.

89. The system of claim 82 wherein said monitor maintains a record of at least one automated blood parameter testing device, at least one monitor, at least one patient, and at least one set of physiological parameters.

90. The system of claim 82 wherein measurement results are stored for trending or later download.

91. The system of claim 82 wherein the system alerts based on predefined levels or ranges for blood parameters.

92. A system for substantially automatically obtaining a blood sample and determining the concentration of at least one analyte comprising:
a monitor;
a central monitoring station; and
a blood parameter testing apparatus, further comprising:
a tube originating from a vascular access point;
a pump mechanism fixedly attached to the tube;
a valve fixedly attached to the tube and located above the pump mechanism;
at least one measurement element;
at least one capillary transport structure;
a needleless port; and
an electronic meter.

93. The system of claim 92 wherein the blood parameter testing device is a blood glucose monitor.

94. The system of claim 92 wherein the blood parameter testing device is in automatic operation.

95. The system of claim 92 wherein the automatic blood parameter testing device is programmable to initiate a periodic sample reading.

96. The system of claim 92 wherein a periodic sample reading is initiated via operator input.

97. The system of claim 92 wherein data is transmitted between the blood parameter testing device and a monitor.

98. The system of claim 92 wherein data is transmitted between a monitor and a central monitoring station.

99. The system of claim 92 wherein said central monitoring station maintains a record of at least one automated blood parameter testing device, at least one monitor, at least one patient, and at least one set of physiological parameters.

100. The system of claim 92 wherein measurement results are stored for trending or later download.

101. The system of claim 92 wherein the system alerts based on predefined levels or ranges for blood parameters.

102. A system for substantially automatically obtaining a blood sample and determining the concentration of at least one analyte comprising:
a monitor;
a central monitoring station; and
a blood parameter testing apparatus, further comprising:
a vascular access point;
a tube originating from the vascular access point;
a pump fixedly attached to the tube;
a first valve fixedly attached to the tube and located above the pump;
a second valve fixedly attached to said tube and located below the pump, wherein said second valve isolates the pump from vascular pressure;
at least one measurement element;
at least one capillary transport structure;
a needleless port; and
an electronic meter.

103. A system for substantially automatically obtaining a blood sample and determining the concentration of at least one analyte comprising:
a monitor;
a central monitoring station; and
a blood parameter testing apparatus, further comprising:
a tube terminating at a vascular access point;
a pump fixedly attached to the tube;
a valve fixedly attached to the tube and located above the pump;
at least one measurement element;
at least one capillary transport structure;
a needleless port;
an electronic meter; and
a sensor.

104. A system for substantially automatically obtaining a blood sample and determining the concentration of at least one analyte comprising:
a monitor;
a central monitoring station; and
a blood parameter testing apparatus, further comprising:
a tube originating from the vascular access point;
a pump fixedly attached to the tube;
a first valve fixedly attached to the tube and located above the pump;
a second valve fixedly attached to the tube and located below the pump;
at least one measurement element;
at least one capillary transport structure;
a needle-less port;
an electronic meter; and
a blood sensor.
